(19) United States
(12) Patent Application Publication
(10) Pub. No.: US 2012/0323326 A1
(43) Pub. Date: Dec. 20, 2012

(54) INSERTION OF ARTIFICIAL/PROSTHETIC FACET JOINTS WITH BALLOTABLE/COMPRESSIBLE JOINT SPACE COMPONENT

(76) Inventor: Frank Harrison Boehm, JR., New Hartford, NY (US)

(21) Appl. No.: 13/347,196

(22) Filed: Jan. 10, 2012

Related U.S. Application Data
(63) Continuation of application No. 13/114,240, filed on May 24, 2011, now abandoned.
Application No. 13/114,240 is a division of application No. 11/224,009, filed on Sep. 13, 2005, now abandoned.

(51) Int. Cl. A61F 2/44

(52) U.S. Cl. 623/17.12

(57) ABSTRACT

A system for replacement of the natural facet joints of the spine is provided. The system is designed to be placed using a minimally-invasive technique, with passage of a unitized prosthesis through a working channel. Alternative embodiments of the prosthesis and method are also provided. The inventor also contemplates a similar system for the cervical and thoracic spine.

### CROSS-REFERENCE TO RELATED APPLICATIONS

This is a continuation of U.S. patent application Ser. No. 13/114,240, filed May 24, 2011 which is a divisional of U.S. patent application Ser. No. 11/224,009 filed Sep. 13, 2005.

### FIELD OF INVENTION

The invention relates to the field of surgery of the spine and, in particular, to surgery involving replacement of the facet joints of the spine with prosthetic implants. The implants are secured to the lamina associated with the inferior articular facet and the pedicle associated with the superior articular facet. A ballottable chamber traverses the joint space. The prosthetic joints may be placed by percutaneous techniques using minimally invasive procedures; endoscopic techniques utilizing slightly more invasive techniques, or “open” surgical technique.

### REFERENCES CITED

| | | |
|---|---|---|
| 5,015,255 | May, 1991 | Kuslich |
| 5,071,437 | December, 1991 | Steffee |
| 5,300,073 | April, 1994 | Ray et al. |
| 5,383,884 | January, 1995 | Summers |
| 5,445,639 | August, 1995 | Kuslich et al. |
| 5,456,722 | October, 1995 | McLeod, et al. |
| 5,491,882 | February, 1996 | Walston et al. |
| 5,571,191 | November, 1996 | Fitz |
| 5,577,995 | November, 1996 | Walker, et al. |
| 5,603,713 | February, 1997 | Aust et al. |
| 5,762,629 | June, 1998 | Kambin |
| 5,792,044 | August, 1998 | Foley, et al. |
| 5,792,044 | August, 1998 | Foley, et al. |
| 5,879,396 | March, 1999 | Walston et al. |
| 5,902,231 | May, 1999 | Foley, et al. |
| 5,954,635 | September, 1999 | Foley, et al. |
| 5,976,146 | November, 1999 | Ogawa, et al. |
| 6,007,487 | December, 1999 | Foley, et al. |
| 6,063,121 | May, 2000 | Xavier, et al. |
| RE36758 | June, 2000 | Fitz |
| 6,095,149 | August, 2000 | Sharkey, et al. |
| 6,113,637 | September, 2000 | Gill, et al. |
| 6,162,170 | December, 2000 | Foley, et al. |
| 6,127,597 | October, 2000 | Beyar, et al. |
| 6,132,464 | October, 2000 | Martin |
| 6,152,871 | November, 2000 | Foley, et al. |
| 6,200,322 | March, 2001 | Branch, et al. |
| 6,419,703 | July, 2002 | Fallin, et al. |
| 6,579,319 | June, 2003 | Goble, et al. |
| 6,610,091 | August, 2003 | Reiley |
| 6,811,567 | November, 2004 | Reiley |
| 6,902,580 | June, 2005 | Fallin, et al. |

### OTHER REFERENCES

Goldthwait, J. E. (1911). The Lumbosacral Articulation. An explanation of many cases of “lumbago,” “sciatica” and paraplegia. Boston Medical Journal, Vol. 64, p. 365-372.
Ghormley, R. K. (1933). Low back pain with special reference to the articular facets, with presentation of an operative procedure. Journal of the American Medical Association. Vol. 101, p. 1773-1777.
Putti, V. (1927). New Conceptions in the Pathogenesis of Sciatic Pain. Lancet. Vol. 2, p. 53-60.
Willliams, P. C. and Yglesias L. (1933). Lumbosacral Facetectomy for Post-fusion Persistent Sciatica. Journal of Bone and Joint Surgery. Vol. 15, p. 579.
Shealey, C. N. (1976). Facet denervation in the management of back and sciatic pain. Clinical Orthopedics, 115, p. 157-164.
Bogduk, N. (1983). The innervation of the lumbar spine. Spine, 8, 286-293.

### BACKGROUND OF THE INVENTION AND RELATED ART

It is estimated that during the course of their lifetime, 65 million Americans will experience one or more significant episodes of back pain, with or without associated radiculopathy. It is the most common reason for adults to seek health care in the United States, and various forms of surgical treatment remain among the most common types of surgical procedures performed. It remains the most common cause of long-term disability from gainful employment. Multiple studies have demonstrated unambiguously that the overall effect of back pain, from this perspective, is almost immeasurable.

At one time, treatment of this disorder was in the hands of a variety of disciplines, including traditional medicine or “allopathic” physicians as well as variety of “alternative” physicians. These included chiropractors, acupuncturists, homeopathist, as well as a variety of less credentialed practitioners and frank charlatans. In sum, back pain was extremely poorly understood at the turn of the 20^{th} century. A variety of potions, recipes, oils, liniments, and mythological regimens that “guaranteed” relief from back pain enjoyed short, but notable tenures as the “panacea” of this all to common disorder.

As a scientific approach began to establish itself, the intervertebral disc began to attract a great deal of attention, particularly as the primary source of symptoms related to the back.

This structure was first recognized as a source of pathology in 1934, when a report by Mixter and Barr appeared in the New England Journal of Medicine describing a herniated disc as a cause of pain in the back and leg (referred to as radiculopathy).

The initial surgical approach called for an incision in the midline of the back over the site of the disc presumed to be diseased. The muscles attached to the posterior elements of the vertebrae are stripped off, exposing the spinous process, lamina, and facet joint. Using a variety of tools, the lamina was then partially or totally removed, and the ligamentum flavum was then removed. The dura and nerve roots were gently retracted medially, and the disc itself could be visualized. The offending fragment was then removed and the incision was closed.

This procedure, either alone or in combination with fusion, was the mainstay of surgical treatment for low back pain and/or radiculopathy for many years. While this had some success, it was well known that many patients did not benefit from this type of surgical intervention. Beginning the late 70's and early 80's, alternatives were sought.

Consequently, there has been a dramatic increase in the technology involved in surgery for disease of the spine over the past two decades. Beginning with the introduction and widespread use of pedicle screws to enhance spinal fusion in the 1980's, there is now a wide armamentarium of devices available to the spinal surgeon.

The reason why a variety of approaches to spinal disorders have been developed is that there have been several key changes in both our scientific understanding, as well as our sense for the patient with back disorders. At one time, it was very common for the majority of physicians to “write off’ patients with back pain leaving these patients with both a sense of abandonment, as well as a sense of desperation, thus precipitating a search for alternative cures. With the advent of transaxial imaging, initially by CT scanning in the late 1970's and subsequently by MRI scanning in the 1980's, a better sense for the complexities of spinal pathophysiology is now appreciated by the average practicing physician. This has led to not only a lower threshold to conduct diagnostic evaluations of such patient's, but also a much lower threshold to refer such patients to spinal specialists. Additionally, it must be stated that changing medicolegal climate over the last quarter century with concerns about malpractice litigation and a sense of practicing “defensive medicine” has also led to an increase in the evaluation and referral patterns of such patients.

However, although such technical advances continue, the current understanding of spinal pathophysiology is only beginning to appreciate the role of all of the complex structures of the spine. As the knowledge base of spinal pathophysiology began to expand, it became obvious that the complex architecture of this articulated column of 24 mobile and 9 fused bones, with their intervening discs, associated facet joints, muscle, tendons and ligaments, there are many different possible “pain generators.” As such, it began to become appreciated that at the laminectomy which had become the standard treatment for any type of spinal disorder, not a panacea; rather it was an appropriate operation for some disorders, and actually, contraindicated for others. Hence, there was at last a logical explanation for the phenomenon of worsening back pain seen in many patients who stated that they actually felt worse after classic laminectomy. While this had long been thought to be a phenomenon was mostly rooted in secondary gain, as the understanding of the pathophysiology of spine disease improved, it became clear that at least a subpopulation of these patients actually were worse after surgery because of the adverse effects of the laminectomy upon their native pathophysiology. Once this was understood, the challenge that developed and still remains is identifying the source of the patient's pathology and pain, and devising surgical treatments that address the specific problem.

One of the structures of the spine that has attracted a great deal of attention as a possible pain generator is the so-called zygapophyseal joint, commonly known as the facet joint. The role of the facet joints in the production of chronic back pain has been noted for many years since the report by Goldthwait in 1911.

It has been noted that each facet joint receives multiple sources of innervation, presumably, therefore, having multiple sources of potential pain transmission. Additionally, Putti in 1927 and Williams and Yglesias (1933) described the facet joint abnormalities that are commonly seen. Ghormley was the first to actually use the term facet joint syndrome in 1933.

In the 1970's, the facet joint became a focus of attention regarding these issues. Reiss successfully described denervation of the facet joints in 1971. This technique was further evaluated and refined by Shealy who introduced the use of radiofrequency thermocoagulation. Multiple other authors have further discussed and refined this technique, including Bogduk, Ogsbury, Simons, Lehman, Pawl, Rashbaum, Sluyter and Mehta.

Despite the extensive literature, the exact role of the facet joints in the overall spectrum of degenerative disease of the spine remains to be clarified further.

Not all authors have been in complete agreement regarding the role of facet disease. Montesano, in the early 80's, published several papers that mitigated very much against the facet joints as a source of pain. These papers became very well established in the orthopedic literature and for more than a decade the facet joints were completely ignored as a source of pain.

With the emergence of pain management clinics, however, the role of the facet joints in the production of back pain has been irrefutably re-established. Causes, however, of both facet arthropathy as well as facet pain itself are still a matter of speculation.

It has always been assumed that facet arthropathy developed as the result of years of microtrauma which is ultimately the consequence of factors such as genetics, heavy labor, poor posture, repeated microtrauma and other, as of yet unexplained factors. It was felt that such factors eventually led to calcium deposits around the facet joints as well as chronic inflammation of the joint proper. This then results in hypertrophy, loss of the stability provided by the joint, and hypermobility. It was further postulated that this ultimately led, in fact, to increased mobility (rather than the classic sense of “stiffness” associated with arthritis) that the increased mobility ultimately resulted in relative motion segment instability. It was felt that this in turn led to back pain as well as possibly nerve root irritation associated with mechanical irritation of the nerve roots as they pass through the foramen.

More recent papers have suggested that the pathophysiology could be even more complicated. Recent studies have evaluated the neurophysiologic and electrophysiologic response of the facet joints to injury. Some papers have suggested that this has kinematic and biomechanical implications.

Other theories have also been postulated. Recently, at the Spine Arthroplasty Society meeting, Ashish has proposed that the build up of nitric oxide within the facet joint could be responsible for some of the pain experienced by these patients. Obviously, these phenomenon need to be further evaluated.

Regardless of the exact cause of facet pain, one fundamentally important concept that has become clearer in recent years is the important biomechanical relationship between the facet joints and the intervertebral disc.

Clearly one important mechanical component regarding the facet joints is the reciprocal relationship with the intervertebral disc. The role of the disc appears to be involved in the governance of movement of the spine, particularly certain movement such as flexion and to a lesser degree lateral rotation and lateral bending. It is felt that the disc is responsible for bearing approximately 80% of the load of the spine while the facet share in approximately 20% of the load bearing. The facets are also responsible for limitation and extension and participate in lateral rotation and lateral bending as well. Recognizing the interrelationship and interplay between the disc and the facet joint becomes apparent that replacement of the disc without attention to the facet joint may create an imbalance. Nevertheless, while a plethora of disc replacement devices have been introduced and contemplated in recent years, few systems exist for the prosthetic replacement of the natural facet joint.

An early attempt to do so was provided by Fitz in U.S. Pat. No. 5,571,191. In this patent he discloses a system by which the facet joints are “capped,” by prostheses. This has not attracted a great deal of interest, and others, including Fallin, in U.S. Pat. No. 6,419,703 has pointed to the shortcomings of such systems. These include difficulties in establishing the correct size of the required cap, failure to relive pain in the setting of advanced osteoarthritis, and the failure of similar systems in other venues (i.e. hip). The system disclosed by Fallin provides a method for replacing the lamina and associated facet joints. Like other systems, discussed below, this requires a substantial surgical procedure.

Martin, in U.S. Pat. No. 6,132,464 also provides for a system that requires attachment to the lamina. It has been commented that the fashion by which this attaches to the lamina would predict substantial variability in the implants, thus being a limiting factor.

Other systems, including that provided by Goble (U.S. Pat. No. 6,579,319) and Reiley (U.S. Pat. No. 6,610,091) require extensive surgical procedures to accomplish implantation. The system disclosed by Reily has undergone initial clinical trials. This system has been the first system to be tested clinically, with the results still pending. Overall, it appears that the experience with such systems is limited, but the need for the surgical replacement of the natural facet joint is becoming increasingly better defined.

Another critical consideration has been the emergence of the artificial disc, or total disc replacement, as a viable surgical option. Early efforts to accomplish this were described at the Charite' hospital in Germany, and subsequently a number of embodiments have been provided by Ray, et al.

Although early evaluations of the use of such prostheses suggest that there is a role for such surgical procedures, it is beginning to be recognized that in some cases, failure of the artificial disc may be related to facet joint pathology. Specifically, the inventors herein postulate that given the reciprocal functional relationship that exists between the facet joints and the disc, it is likely that in a number of cases, disease of the facet joints and the intervertebral disc is most likely coexistent. In such settings, it is readily postulated that replacement of the disc without attention to the [diseased] facet joints will result in failure of the prosthesis.

The facet joint prostheses that have thus far been provided, appear to satisfy the biomechanical requirements placed upon such a prosthesis. These requirements would include the ability to share in the biomechanical load of the spine, particularly the lower spine. This has been estimated to be approximately 2000N in the erect individual, and it is thought that the disc bears approximately 80% of this physiological load while the facet joints bear approximately 20%, or approximately 400N.

There are also shear and strain forces placed upon the facet joint, and, therefore, upon any prosthesis that might be implanted as a replacement of the facet joint. Additionally, the joint participates in the limitation of movement in flexion, lateral rotation, lateral bending, and, in particular, extension. Such a prosthesis would, necessarily, have to perform such functions.

Therefore, a need exists for a system of facet joint replacement that is minimally-invasive yet able to satisfy the biomechanical requirements of the natural facet joint.

### SUMMARY OF THE INVENTION

The invention is made bearing the above needs in mind, and in accordance with those needs, one aspect of the invention provides for a system by which the surgeon may identify the facet joints in an efficient, accurate, and technically facile manner. Such a system has been provided for by the inventors in a previous application. As such, the surgeon can utilize fluoroscopy in conjunction with purpose-specific templates that are laid against the back of the patient. This template is impregnated with a radioopaque marker that simulates the outline of the lateral profile of the spine in the posteroanterior projection. Lateral to this outline is a raised aperture that will direct the trajectory of a needle/guide pin that has been passed through it. Aligning the outline on the template with the radiographic image of the spine will allow the surgeon to identify an entry point through which a needle/guide pin may be passed into the facet joint without difficulty. The position of the needle is then confirmed using radiologic techniques.\

In another aspect of the invention, when an adequate position of the needle has been established, a series of dilators are passed over the needle ultimately defining a pathway for passage of a working channel. The working channel is unique, as described in a previous application, insofar that the shape of the working channel is ovoid, or elliptical in shape. This device is tubular is structure, and demonstrates a leading end, a long axis, and a trailing end. The leading end is passed through the incision and brought against the target facet. The long axis then connects the leading end to the trailing end, the trailing end being the site whereupon the surgeon may be provided with access to the interior of the working channel for passage of appropriate additional devices as well as insertion of the prosthesis. Furthermore, in the preferred embodiment, the working channel is provided with a modification of its leading end in that there is an extension seen arising from the lateral aspect of the leading end. This extension is designed in such a fashion that this extension can be brought along the lateral aspect of the superior articular process and docked against the junction of the superior articular process and the transverse process. This design will add additional stability to the working channel during the balance of the procedure.

In yet another aspect of this invention, the diseased facet joint is removed with a burr, box chisel, or by some other means. The preferred embodiment favors the use of a burr, in a fashion similar to the previous application provided by the inventor. As such, the preparation for removal of the facet joint will involve similar steps. These steps include proper identification of the facet joint, passage of a series of dilators to separate the muscular attachment to the facet joint, passage of a working channel through which a burr or other instrument can be used to remove the facet joint. Removal of the facet joint includes removal of both the superior and inferior articular [processes] components of the joint. This maneuver leaves behind an entry into the pedicle as well as an area along the lateral lamina for attachment of the joint prosthesis.

In the preferred embodiment, after removal of the natural facet joint, the prosthesis is passed down so that it may engage the entry point into the pedicle. The prosthesis is composed of a leading end and a trailing end. The leading end, in turn, is represented by a screw which shall be passed through the target pedicle and anchors the prosthesis into the vertebral body. The leading end of the screw may be designed in such a way that it can either be self-tapping and/or self-drilling. Alternatively, a drill and tap may be used in the classic fashion for insertion of a screw into bone.

The trailing end of the prosthesis is represented by a multi-component structure which reproduces the movement accorded to the motion segment by the facet joint to be replaced. These components include a superior articular replacement component, as well as a ballottable chamber and a laminar component. The superior articular replacement component is similar in size and configuration to a normal superior articular process. The trailing end of the screw is contained within a chamber that is located within the superior articular replacement component. Specifically, the trailing end of the screw is secured within a chamber that is located within the central portion of this superior articular process replacement. The trailing end of the screw is designed to accommodate a Philips-type screwdriver, a regular or flat screwdriver, an Allen wrench, or any other similar drive mechanism. The trailing end of this chamber is continuous with an aperture on the superior surface of the superior articular replacement, and this aperture provides access of such a screwdriver or other drive mechanism to the trailing end of the screw. At the leading end of the chamber, the trailing end of the screw, which is noted to be slightly enlarged in diameter when compared to the diameter of the shaft of the screw, interfaces with an engagement mechanism. In the preferred embodiment, this is represented by a narrowing of the chamber, so that as the screw passes into the pedicle, a point is reached whereby the trailing end of the screw becomes secured against the walls of the chamber. A number of embodiments are contemplated, with the primary goal to provide a system by which the screw can be initially rotated so that it may be passed into the pedicle. When an adequate depth has been achieved, in the preferred embodiment, the trailing end of the screw is brought against the walls of the chamber and irreversibly secured into position. One or more washers/bushings may be incorporated into the construct of the engagement mechanism to achieve this. A locking or securing “cap” or plug may be passed into the trailing end of the chamber to further secure the screw.

On the medial side of the superior articular process replacement there is found a discoid, flattened area which shall serve as the base of attachment for a ballotable chamber which may be filled with fluid, air, a granular substance or any other substance. This chamber then subserves the main functions of the mobility of the facet joint. The fluid nature of this chamber will allow for rotational as well as shear movement, and extension of the chamber as well as compression. On the medial side of this ballottable chamber is yet another discoid base that secures to the chamber. This discoid base in turn is irreversibly coupled to a vertical extension of the most medial component of the prosthesis, that component being referred to as the laminar component. The laminar component has a vertically-oriented extension which attaches to the ballottable chamber. The vertical extension then forms an angle with a somewhat horizontally-oriented extension which is designed to lie against the lateral aspect of the lamina. Another embodiment of the laminar component replaces the angle between the horizontal and vertical components with a single, smooth curvilinear embodiment. This is designed to more easily conform to the area of the lamina that has been removed by the burr. In either embodiment, the surface that interfaces with the bone would ideally be roughened or in some other fashion finished, coated, or treated so as to promote bony ingrowth. This is secured to the lamina by a plurality of screws, pins, or other similar securing devices. The screws or other securing devices may be oriented to secure into the lamina, or conversely, may be directed to be passed through the base of the spinous process. In yet another alternative embodiment, the screws may be passed through the base of the spinous process and secured with a bolt or nut on the exterior surface of the contralateral lamina. These screws are secured into place by right angled screwdrivers. Alternatively, panels in the working channel may be removed to allow access to the proper angle for insertion.

On the medial aspect of the ballotable chamber is yet another discoid surface which is anchored to the chamber. This discoid surface is in turn on the lateral aspect of the component that is secured to the lateral aspect of the lamina.\

The presence of the ballotable chamber provides movement in a variety of planes and accounts for the shear and stress movements typically mitigated by the facet joint.

In another embodiment, the inflatable chamber is replaced by a 2-piece mechanical chamber that is designed to limit extension; at the same time this mechanical chamber is fashioned in such a way that it will not constrain shear movement of the joint nor will it constrain lateral rotation or lateral bending. This is achieved by the inner design of the mechanical chamber, which is composed of two ramps that are spiraling in opposite directions. Furthermore, there is a space between the high points of both ramps such that this space closes and is eliminated in extension, but opens in flexion and remains neutral in lateral bending and lateral rotation. Furthermore, the space will again open with shear movements of the facet joints. This embodiment is, again, unitized and inserted using the same sequence of steps that are used to insert the preferred embodiment outlined above—namely, the target facet joint is radiographically identified; a guide pin is passed into the joint; dilators and a working channel are then passed into place; burr is used to remove the joint; and the prosthesis is inserted. The prosthesis can be secured to the lamina using the preferred or alternative embodiments outlined above.

### BRIEF DESCRIPTION OF DRAWINGS

FIG. 1A Posterior view of the superior and inferior vertebrae comprising a motion segment; target facet joint joins the posterior aspects of these 2 vertebrae.

FIG. 1B Lateral view of the superior and inferior vertebrae comprising a motion segment; target facet joint joins the posterior aspects of these 2 vertebrae.

FIG. 2A Frontal view of an embodiment of the prosthetic device.

FIG. 2B. Frontal view of alternative embodiment of the prosthetic device.

FIG. 3A Exploded perspective view of the facet joint prosthetic device;

FIG. 3B Exploded frontal view of the facet joint prosthetic device.

FIG. 3C Frontal view demonstrating relationship of trailing end of screw to chamber and engagement mechanism.

FIG. 3D Frontal view of enlarged view of superior articular replacement; the shaft and trailing end of screw approaching the engagement mechanism can be seen.

FIG. 3E Elevated perspective view of the superior articular replacement.

FIG. 4 Posterior view of the pair of lumbar vertebrae showing the position of a series of dilators, each successive dilator being slightly larger than the previous dilator, passed over the guide needle during creation of the working channel

FIG. 5A Transparent template sheet with radioopaque outline of lateral profile of spine. Apertures to direct guide pin are seen laterally to the profile.

FIG. 5B Template laid over back of patient. The radioopaque guide on the alignment template is matched to the lateral profile of the spine.

FIG. 6 Posterior view of the pair of lumbar vertebrae with a guide pin having been placed adjacent to a facet joint to be replaced.

FIG. 7 Posterior view of lumbar vertebrae with dilator having been passed over guide pin.

FIG. 8 Posterior view of pair of lumbar vertebrae with working channel having been passed over dilator and guide pin.

FIG. 9 Posterior view of working channel in place with needle removed.

FIG. 10A Posterior view of hand-driven burr, having been passed through the working channel, removing facet joint.

FIG. 10B Posterior view of the pair of vertebrae after a cutting tool has removed the facets to be replaced.

FIG. 11A Transaxial view with working channel in place.

FIG. 11B Transaxial view with working channel in place. Facet has been removed.

FIG. 11C Transaxial view with drill having been passed through working channel and into pedicle.

FIG. 11D Transaxial view with tap being passed through working channel into pedicle.

FIG. 12A An embodiment of a hand-driven device to insert the prosthetic device into place.

FIG. 12B The leading end of the insertion device is engaged with the trailing end of the screw; frontal view.

FIG. 13 Posterior view of prosthetic device being passed through the working channel in preparation for insertion.

FIG. 14A Transaxial view of an embodiment of the device in place; screws to secure laminar component are within the lamina.

FIG. 14B Transaxial view of an embodiment of the device in place; screws to secure the laminar component are passed across base of spinous process.

FIG. 14C Transaxial view of alternative embodiment of the laminar component; device in place.

FIG. 15 Exploded view of alternative embodiment of the joint disc.

FIG. 16A Schematic of alternative embodiment of joint disc; this is a frontal view of the disarticulated mirror image disc joint components. The ramps and points of maximum elevation are illustrated; arrows indicate direction of rotation of the components.

FIG. 16B Schematic of lateral view of alternative embodiment of joint disc.

FIG. 17A Schematic showing frontal view of alternative embodiments of discoid surface and discoid base; the horizontal bars can lock preventing excessive extension.

FIG. 17B Schematic of frontal view of discoid surface and discoid base in place around ballottable joint disc; the horizontal bars have locked.

### DETAILED DESCRIPTION OF DRAWINGS

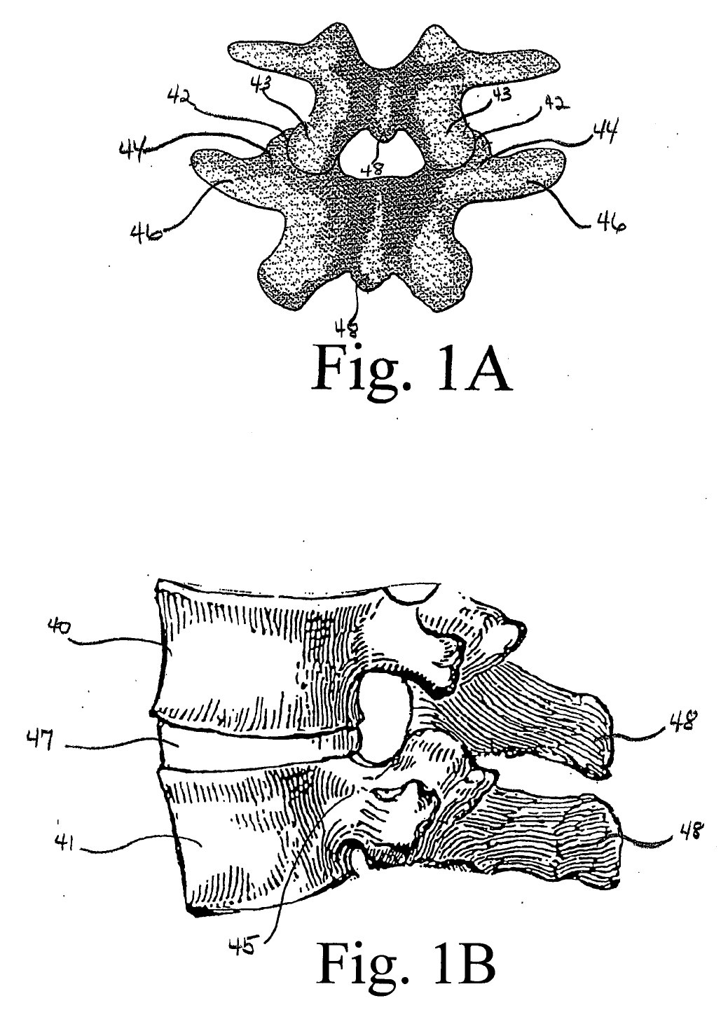

Referring now to the drawings, in which like reference numerals identify similar or identical elements throughout the many views. Turning now to FIG. 1, in order to better understand the invention and its implantation during a surgical procedure, it would be most helpful to have a better understanding of the appropriate anatomy. In FIGS. 1A and 1B, are demonstrated a posterior (1A) and lateral (1B) views of two vertebrae comprising of a motion segment. The superior vertebral body (40) and inferior vertebral body (41) are best seen on the lateral (FIG. 1B) view above and below the intervening disc. The target facet joint (42) lying between the posterior elements of the two vertebrae, and is comprised of the superior articular process (43) and the inferior articular process (44). The target pedicle (45) is associated with the inferior articular process and hence the inferior vertebral body (41) of the target and motion segment.

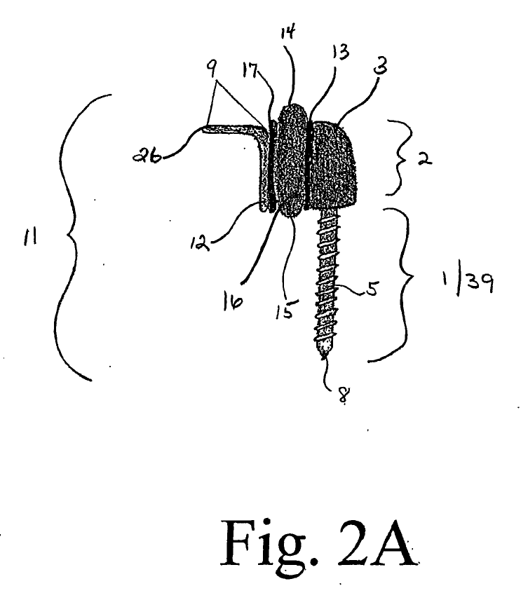

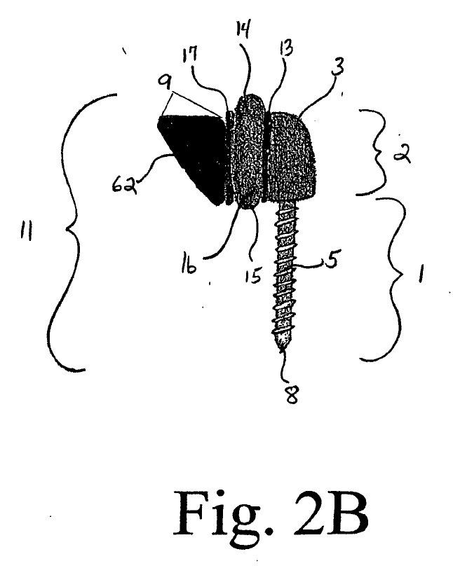

FIG. 2A demonstrates a frontal view of the prosthetic device (11). In the preferred embodiment, the leading end (1) and the trailing end (2) of the device are shown with the leading end consisting of a screw (39) which itself consists of a leading end (8) and a shaft (5). The leading end (8) and shaft (5) are passed through the long axis of the target pedicle (Not shown in this figure). Again the target pedicle is being defined as the pedicle of the more caudal vertebrae in the motion segment. The trailing end (2) consists of multiple components, including the superior articular replacement (3) which serves as the replacement for the superior articular process (Not shown in this figure). On the medial aspect of the superior articular replacement (3), there is found a discoid shaped base (13) which serves as a connection between the superior articular replacement (3) and the remainder of the trailing end of the prosthesis (2). On the medial side of the discoid base (13) is found a ballottable chamber (14). In the preferred embodiment, this ballottable chamber has a flexible outer surface (15) composed of latex, polymer, silicone, or any other substance either previously used in the art or not. This flexible outer surface (15) contains within it, a ballottable fluid or gelatinous center (16). It is the flexibility provided by this ballottable center which permits the joint a multi-directional movement and also provides shear and torsional vector management to the prosthesis. The medial aspect of the ballottable chamber (14) is in turn attached to another discoid base (17). This is connected to the vertical extension (12) of the laminar component (9). There is also seen in this projection, a horizontal extension (26) of the laminar component (9). This horizontal extension (26) shall be a point of attachment to the lamina.

FIG. 2B demonstrates a frontal view of an alternative embodiment of the prosthetic device (11). Specifically, this image demonstrates an alternative embodiment of the laminar component (9). In this embodiment, rather than a distinct vertical extension (12) and horizontal extension (26), these components are replaced by a unitized component (61) that demonstrates a curvilinear surface (62) of various thicknesses which is connected to the discoid base (17). The curvilinear component (62) has a convex surface (63) which is brought against the cut surface of the lateral aspect of the lamina (59). This unitized component (61) is again secured to the lamina (59) by securing screws (58) which are passed through a plurality of holes (64) in the unitized component (61).

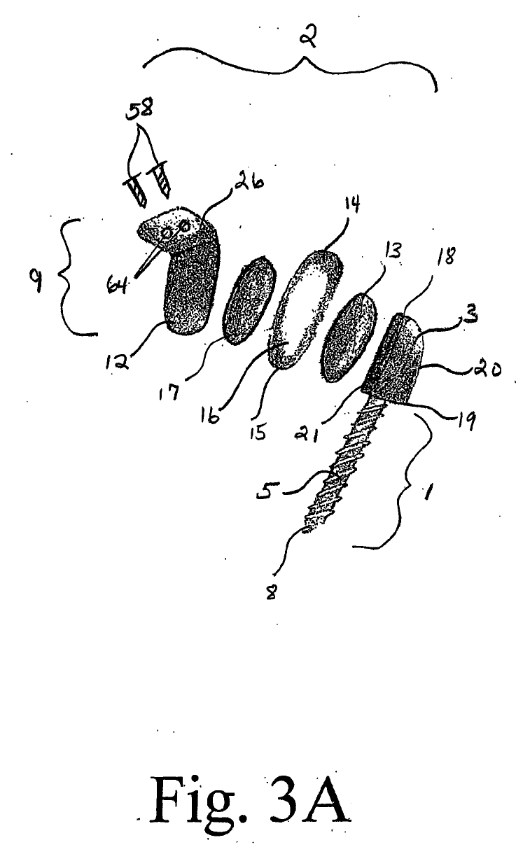

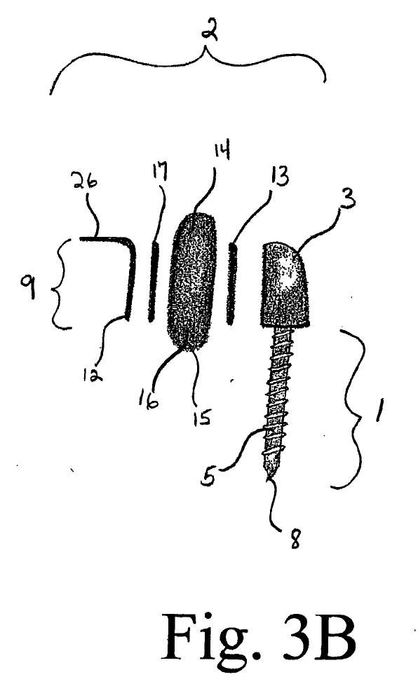

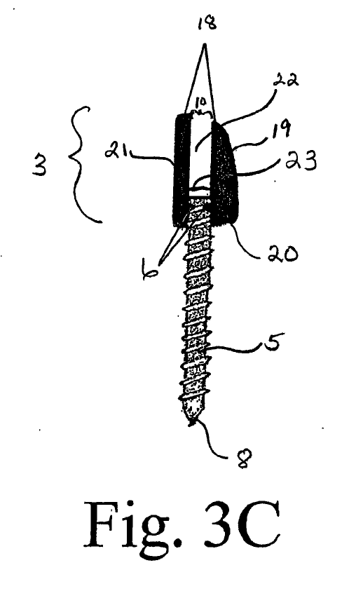

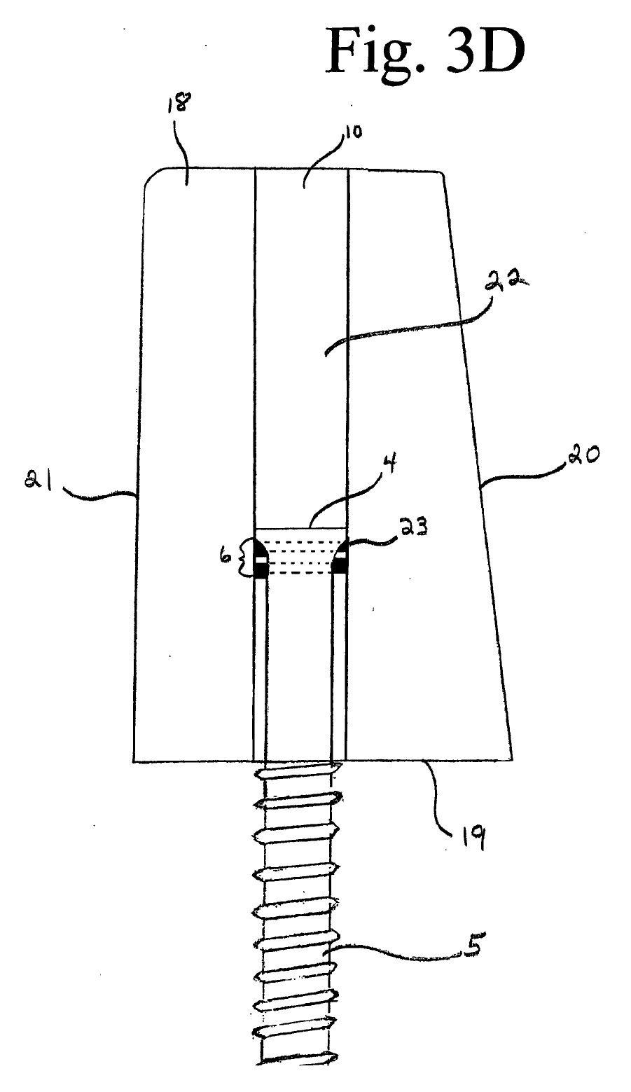

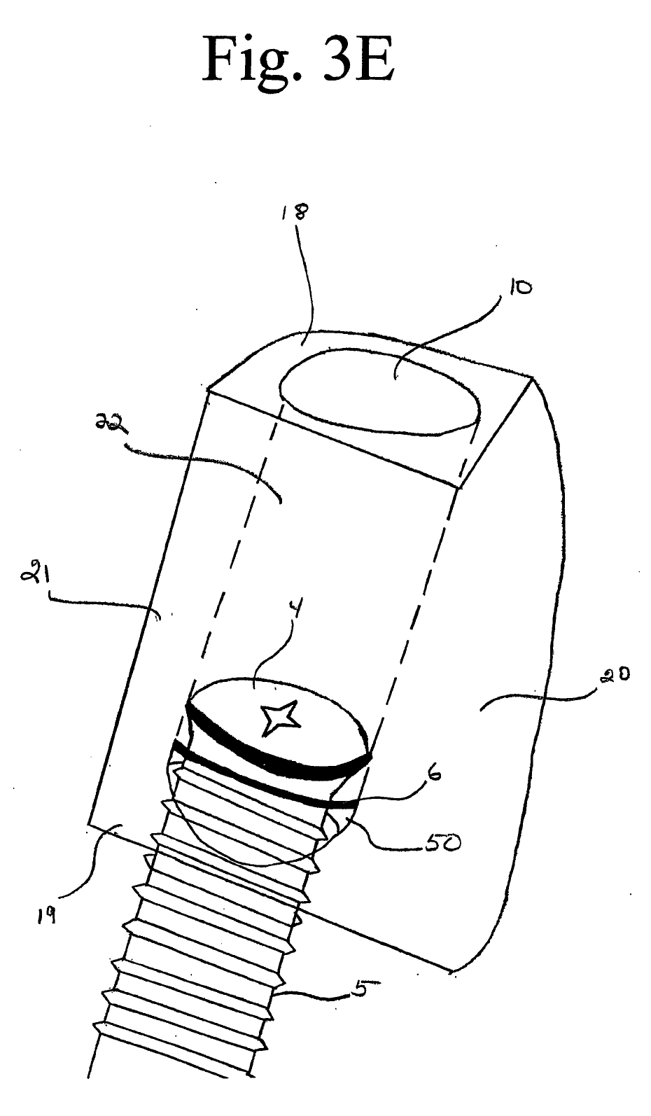

As demonstrated in FIGS. 3A and 3B, exploded views of the prosthetic device itself, there are additional components to the device. The leading end of the device (1) itself has a leading end or tip (8) and a shaft (5). In a similar fashion, the trailing end (2) is composed of multiple additional components including the superior articular replacement (3). This is an oblong of ovoid structure and on it is found a superior surface (18), an inferior surface (19), a lateral surface (20) and a medial surface (21). On the medial surface (21) of the superior articular replacement (3) is found a discoid base (13). This discoid base (13) serves as an attachment for a ballottable disc-shaped structure (14), herein known as a joint disc which is irreversibly attached to the discoid base (13). The ballottable joint disc (14) is of various thickness and width and is composed of a flexible outer skin (15) and a liquid or gel center (16). The liquid or gel center (16) allows for movement of this joint in all planes and serves as the functional replacement of the facet joint. On the medial side of the joint disc (14), is irreversibly attached to yet another discoid base (17). This discoid base (17) is connected or attached to a vertical extension (12) of the laminar component (9). There is also a horizontal extension (26) of the laminar component (9). This horizontal extension (26) is secured to the lamina (Not shown) by securing screws (58) which pass through a plurality of holes (64).

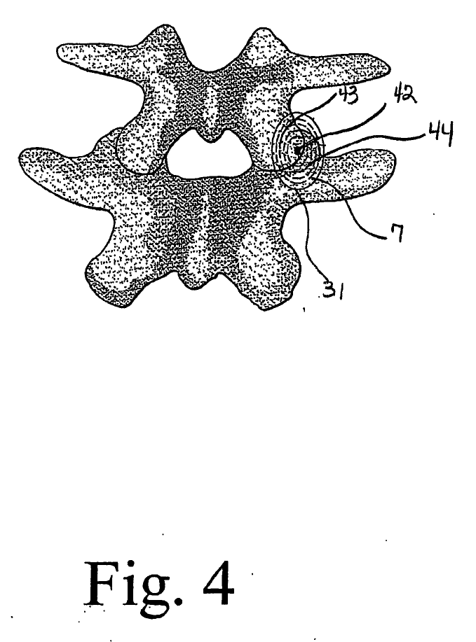

In FIG. 3C, it can be seen that the superior articular replacement (3) is an oblong or ovoid shaped structure provided with a superior surface (18) and an inferior surface (19) lateral surface (20) and medial surface (21). Furthermore, there is an aperture (10) which is provided in the superior surface (18). This aperture (10) is continuous with a chamber (22), of variable length, which passes through the body of the superior articular replacement (3). Within the chamber (22) lies the trailing end of the screw itself (4) which is provided with an insertion for a Phillips type screwhead, a regular type screwhead, Allen wrench, or any other type of drive mechanism. Furthermore, lying in close proximity to the trailing end of the screw (4) in the undeployed state is found an engagement mechanism (6) which is found at the leading of the chamber (22). This is continuous with a second aperture (50) on the inferior surface (19) of the superior articular replacement (3). In the preferred embodiment, the diameter of the trailing end of the screw (4) demonstrates a slight enlargement with respect to the diameter of the shaft of the screw (5). Conversely, in the engagement mechanism (6), there is a slight diminution in the diameter of the chamber (22) with respect to the leading end of the chamber so that this can form a coupling with the trailing end of the screw (4). There may be one or more washers and/or bushings present in this engagement mechanism (6) to assist in securing the trailing end of the screw (4) to the engagement mechanism (6). The design is such that when the screw (39) is being passed into the pedicle (45), the engagement mechanism (6) will initially permit the screw (39) to rotate freely so that it may be advanced into the pedicle (45) without rotating the prosthetic device (11) or becoming secured prematurely to the engagement mechanism (6). However, as the screw (39) has been fully passed through the target pedicle (45), the trailing end of the screw (4) will engage the engagement mechanism (6), ultimately serving to lag the prosthetic device (11) against the osseous surface at the site of the entry point into the target pedicle (45). There may be one or more washers also located in the region of the engagement mechanism (6). This relationship is provided in such a fashion that as the screw (39) is passed into the pedicle, the trailing end of the device (2) is brought down into the site of the intended facet joint replacement, as seen as the stippled area (7) in FIG. 4. This specialized relationship is further illustrated in FIGS. 3D and 3E. FIG. 3D demonstrates a frontal profile of the superior articular replacement component (3). This is noted to have a superior surface (18), a lateral surface (20) and as inferior surface (19) and a medial surface (21). On the superior surface (18), there is an aperture (10) that leads into a chamber (22) in which the trailing end of the screw (4) is contained. At an arbitrary point in the chamber (22) there is an engaging mechanism (6), as well as an engaging ring (23). As the leading end of the screw (4) and shaft (5) are passed into the bone, the trailing end of the screw (4) is allowed to rotate freely within the chamber (22). However, when the inferior surface of the replacement (19) is brought against the bony entrance to the pedicle, the trailing end of the screw (4) becomes irreversibly secured within the engagement mechanism (6). The shaft of the screw is noted to pass through an aperture (50) in the leading end of the chamber (22) at its junction with the inferior surface (19). FIG. 3E demonstrates an elevational view further illustrating this mechanism.

In FIG. 4 is seen a view of the posterior aspect of the intended site of facet joint replacement (42). Although the stippled area (7) is seen unilaterally, it is recognized that in most cases, this would be performed bilaterally. The stippled area (7) can be seen extending over the superior articular process (43) as well as the inferior articular process (44). The stippled area (7) is noted to be ovoid in shape representing the preferred embodiment of the working channel (31).

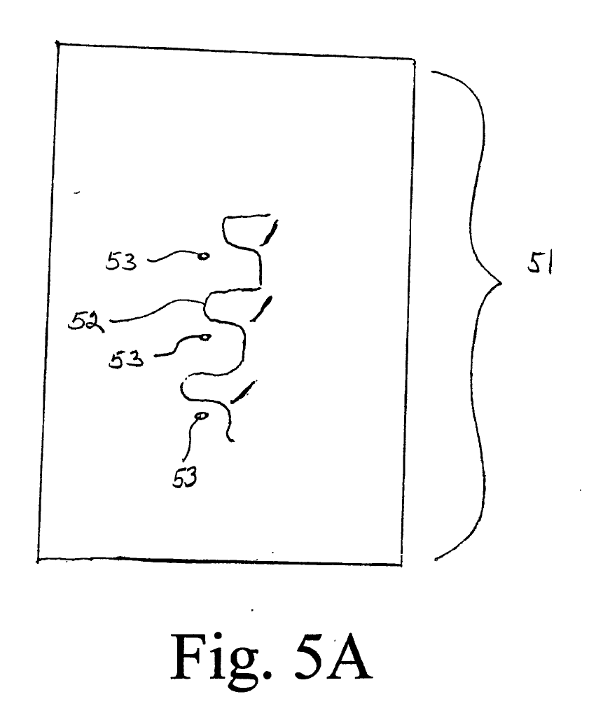

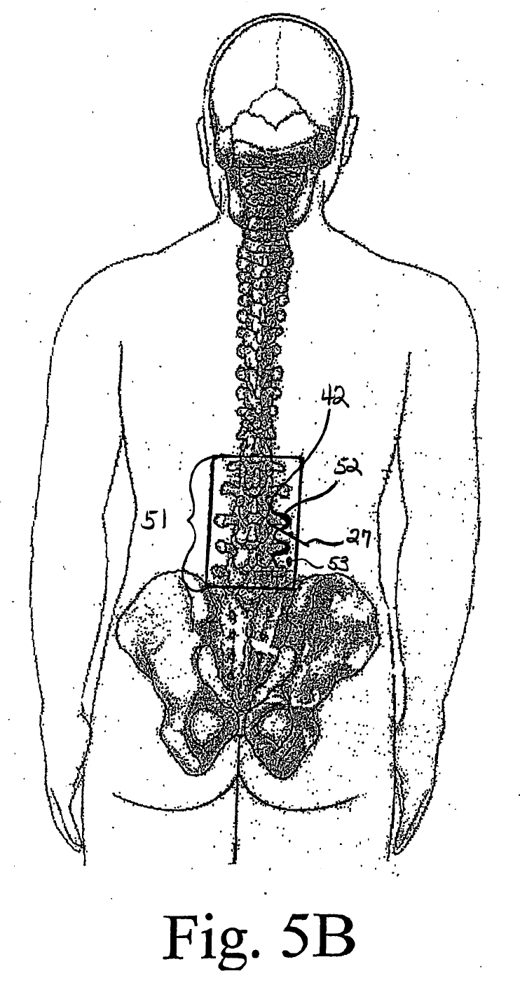

FIG. 5A demonstrates a transparent, sterile sheet (51) which is impregnated with a radioopaque image representing the lateral profile (52) of the spine as seen in the posteroanterior view. Lateral to the profile (52) of the spine is a raised aperture (53). The aperture (53) is used to direct the trajectory of a guide pin (not shown in this figure) as it is directed towards to the target facet joint (not shown in this figure).

In FIG. 5B, this sheet (51) is laid against the back of the prone patient. It is assumed that an image-guided system is being used to demonstrate an image of the patient's spine in the posteroanterior view. As such, the radioopaque profile (52) is aligned with the image-guided projection of the spine. The raised aperture (53) in the sheet (51) then directs the guide pin (27) into the facet joint (42).

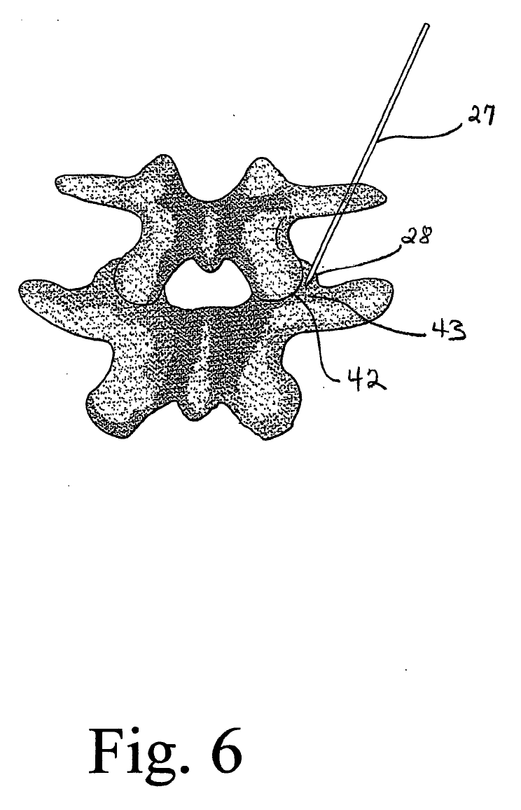

FIG. 6 is a posterior view demonstrating the guide pin (27) having been passed successfully, with its leading end (28) resting against the superior articular process (43) in the immediate proximity of the joint to be replaced (42).

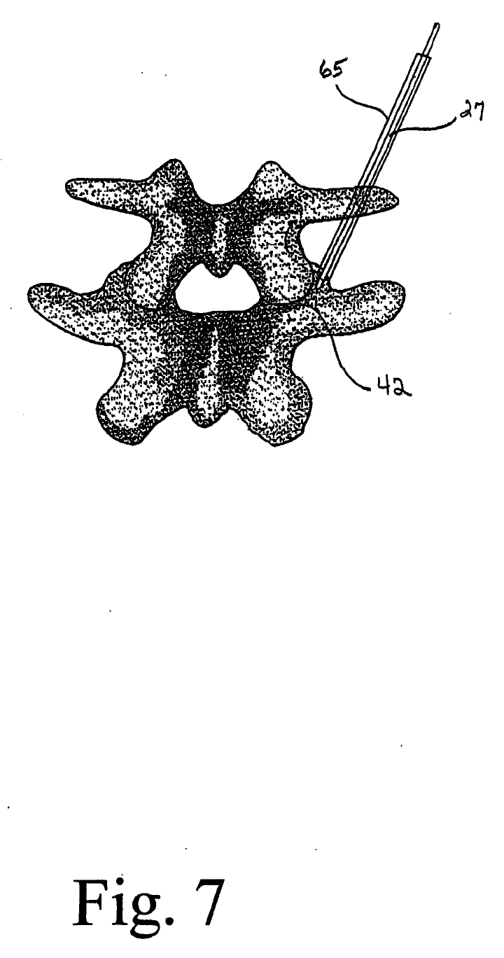

FIG. 7 is a posterior view demonstrating the guide pin (27) having been successfully passed into the region of the target facet (42). A dilator (65) has been passed over the guide pin (27) in order to dilate the soft tissues along the tract from the skin entry point to the area of intended facet replacement (42).

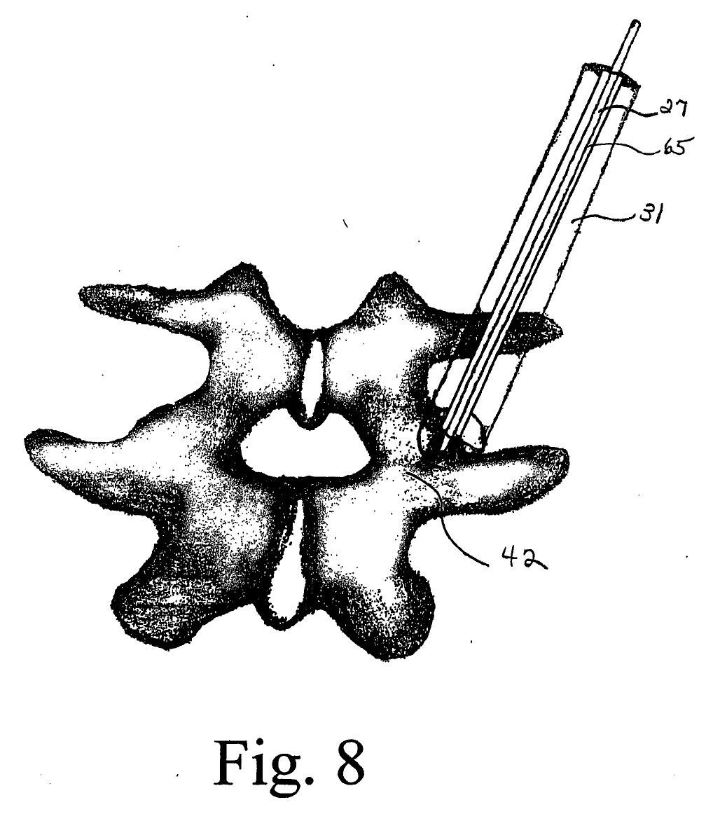

FIG. 8 also demonstrates a posterior view of the lumbar vertebrae with the working channel (31) having been passed over the guide pin (27) and dilator (65) complex. The passage of one or more dilators (65) have adequately distracted the soft tissues surrounding the proposed tract between the skin and the intended facet replacement (42). It is recognized that other forms of distractors including expandable distractors as well as multiple distractors that expand in a centrifugal fashion are also able to accomplish this goal. This will allow easy passage of the working channel (31).

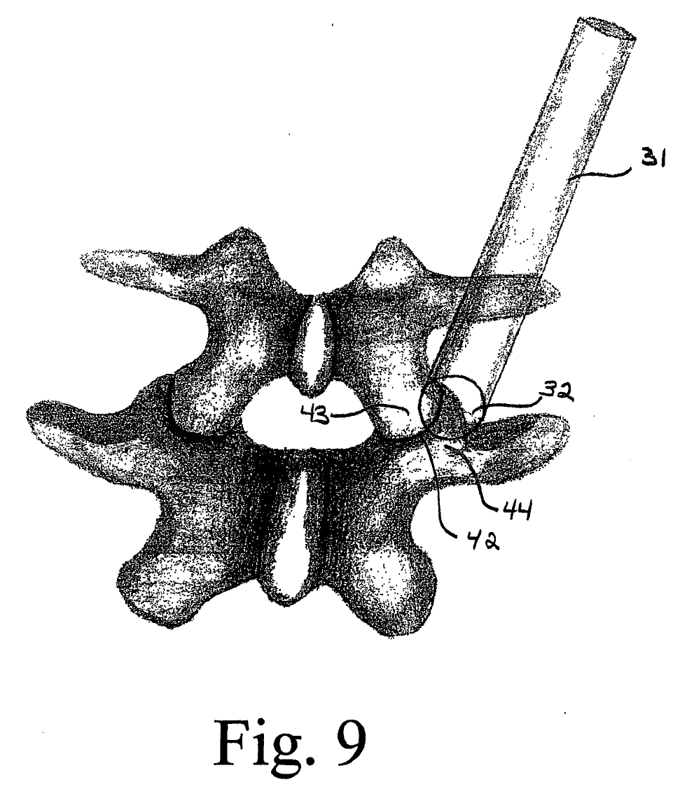

As demonstrated in FIG. 9, a posterior view of the vertebrae is seen with the working channel (31) in place. The guide pin and dilators have been removed. The leading end (32) is seen extending over the superior (43) and inferior (44) articular processes. In the preferred embodiment, the leading end (32) is configured with an extension (not seen in this figure) to improve the fashion with which it seats against the superior articular process (43) and entry point to target pedicle (not seen in this figure). Such a configuration of the leading end (32) of the working channel (31) will allow for greater facility in burring out the facet (42) as well as accessing the target pedicle (45). It is to be recalled that the target pedicle serves as the anchor for the leading end of the prosthetic device.

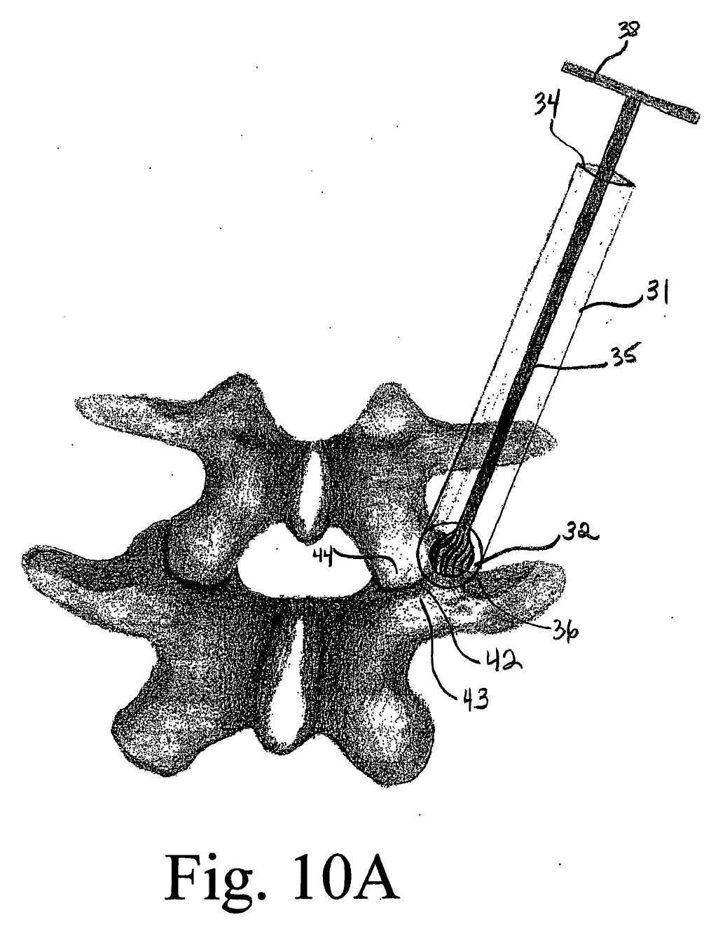

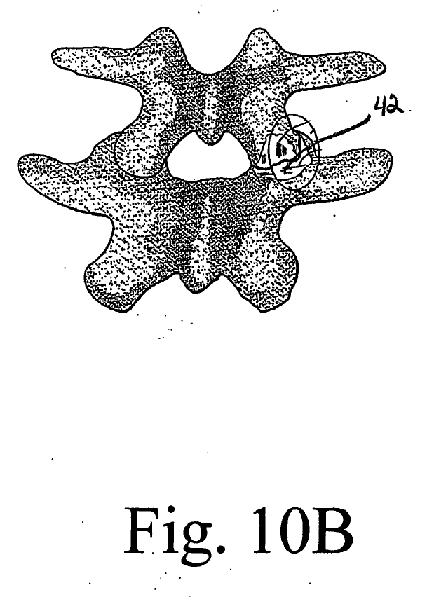

FIG. 10A demonstrates a posterior view with the working channel (31) in place. A hand-driven round burr (35) has been passed down to remove the target facet joint (42). The leading end of the burr (36) can be seen resting against the target facet joint (42). As the handle (38) is rotated, the joint as well as the superior (43) and inferior (44) articular processes are removed by the cutting grooves of the burr (36). Having completed this action, the target facet joint (42) is now prepared for insertion.

As seen in FIG. 10B, a posterior view of the spine is demonstrated with the target facet joint (42) having been removed.

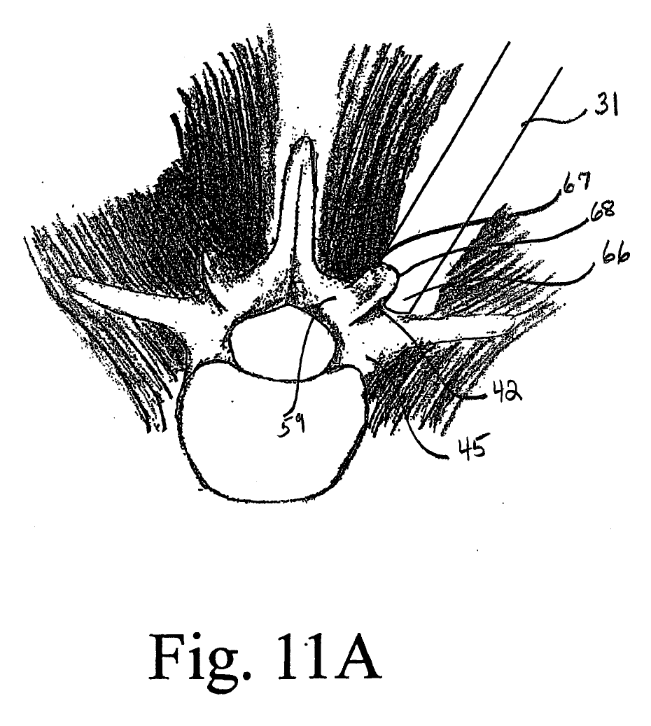

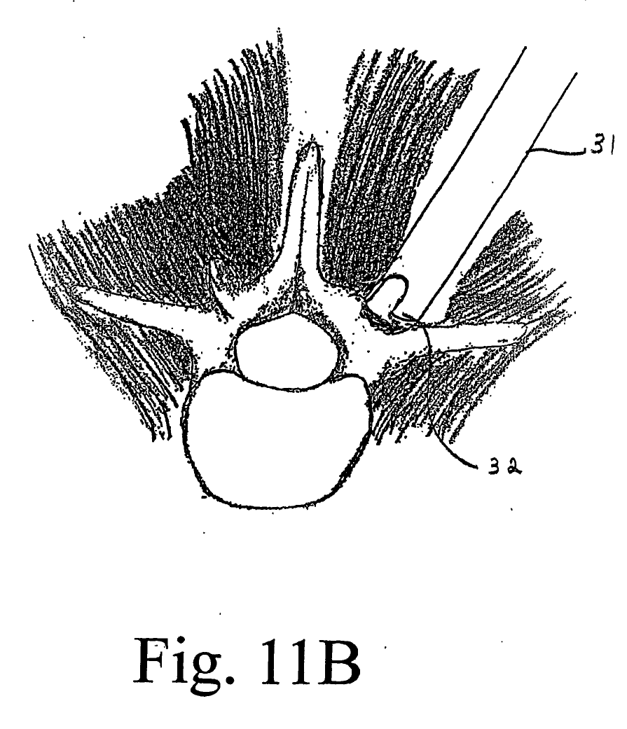

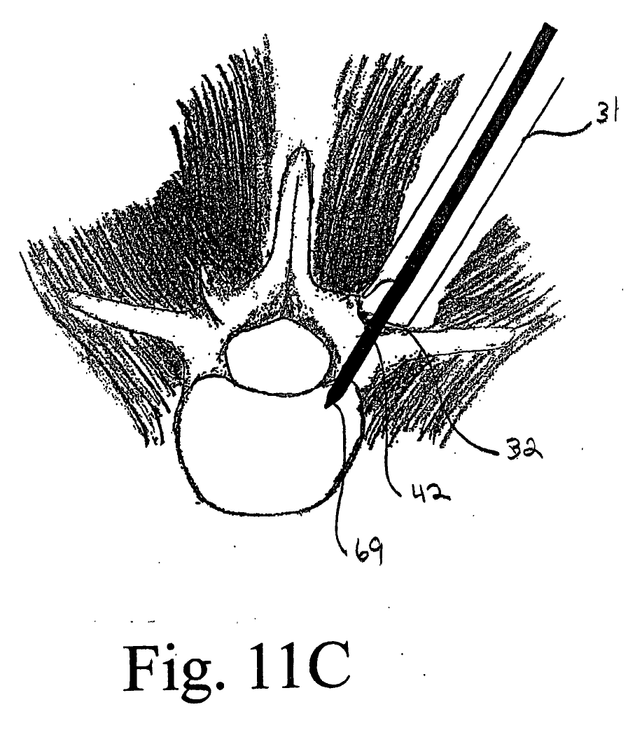

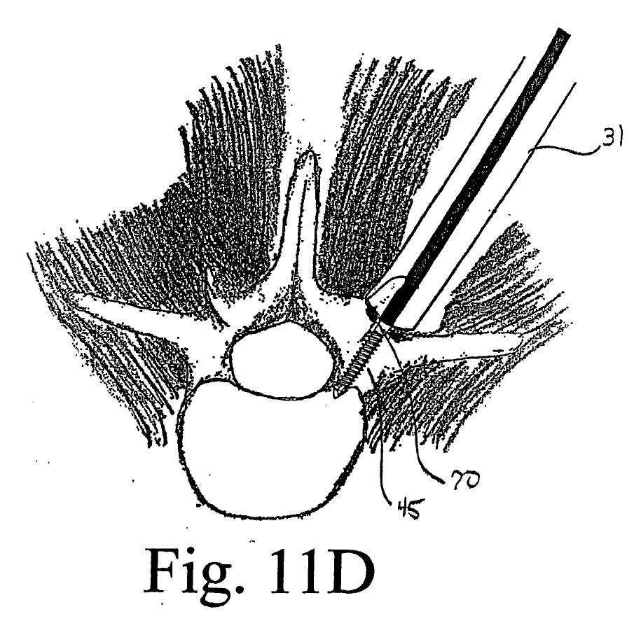

Now, in FIG. 11A is demonstrated a transaxial view with the working channel (31) in place. An extension (66) of the working channel (31) on the lateral aspect allows the channel to be seated over the entry point to the target pedicle (45) and also extend over the target facet (42). A second extension (67) allows for the working channel (31) to be securely placed against the lamina (59). A cutout (68) at the end of the preferred embodiment of the working channel (31) allows for the working channel (31) to be brought against the facet joint (42).

In FIG. 11B, the facet has been removed in this transaxial view. The configuration of the leading end of the working channel (32) allows the working channel (31) to remain stable after removal of the facet. The area of bony removal can be seen inferior to the leading end of the working channel (32). The site has now been prepared for passage of the leading end of the device.

FIGS. 11C and 11D demonstrate transaxial views with the passage of the drill (69) and the tap (70) into the target pedicle (45). This prepares the site for the passage of the leading end of the device (1) into the target pedicle (45). In an alternative embodiment, the leading end of the screw (8) can be configured so that it is self-tapping and/or self-drilling, hence eliminating the need for these steps.

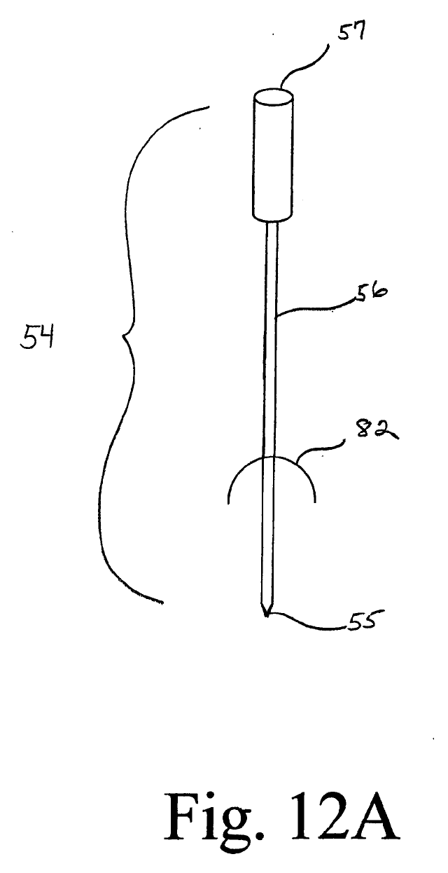

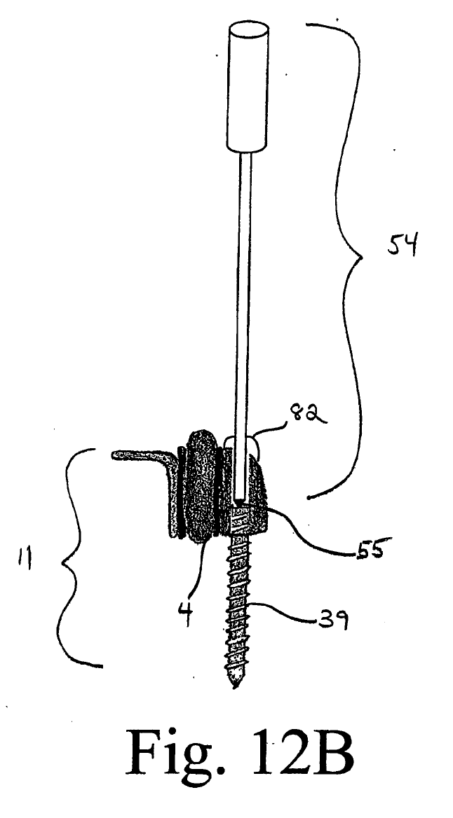

In preparation for passage of the prosthetic device (11), an insertion tool (54) is now introduced into the procedure. As seen in FIG. 12A, the insertion tool (54) is itself composed of a leading end (55), a shaft (56) and a trailing end (57). The leading end (55) may demonstrate various embodiments including a Phillips head configuration, a straight screwdriver head, an Allen wrench type configuration or any other similar drive mechanism. Similarly, the trailing end (57) may encompass any embodiment that allows for manual or power rotation of the screwdriver (54). A special feature is a cradle (82) which stabilizes to the superior surface of the superior articular replacement. This cradle (82) maintains the position of the prosthesis while the insertion is being undertaken.

FIG. 12B demonstrates the screwdriver (54) engaging the prosthetic device (11). The leading end of the screwdriver (55) is designed to reversibly couple with the trailing end of the screw (4) within the chamber (22) of the superior articular replacement (3). The cradle (82) is arranged so that the screw (39) can be advanced into the target pedicle (45) without rotation of the entire prosthetic device (11).

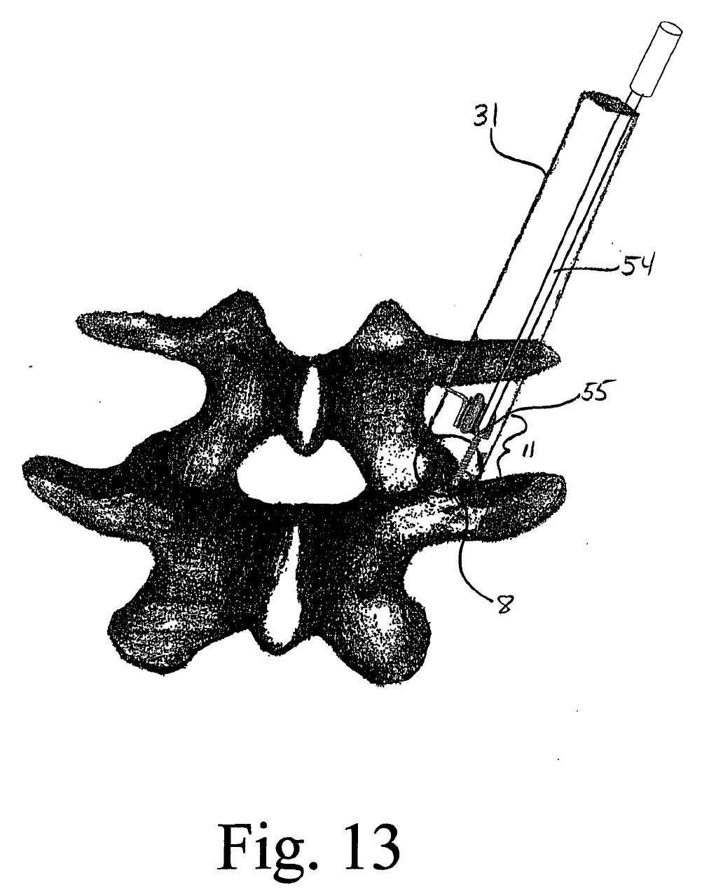

FIG. 13 is a posterior view with the working channel (31) in place and the prosthetic device (11) being passed through the working channel (31) on the leading end (55) of the screwdriver (54). The leading end of the screw (8) is positioned within the working channel (31) to engage the entry point of the target pedicle (not shown in this figure).

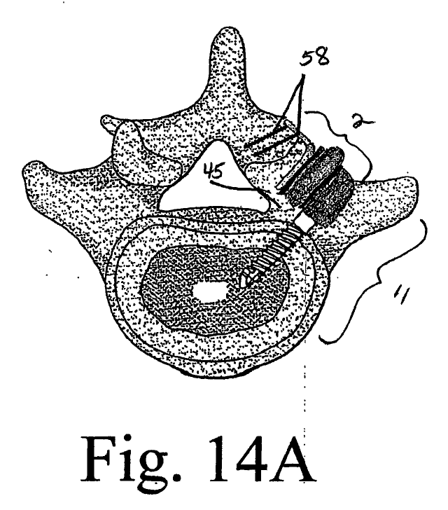

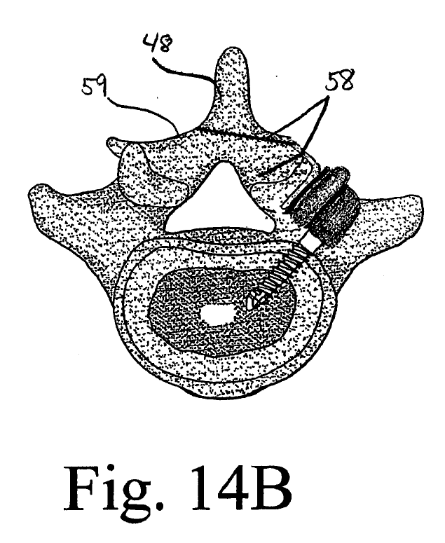

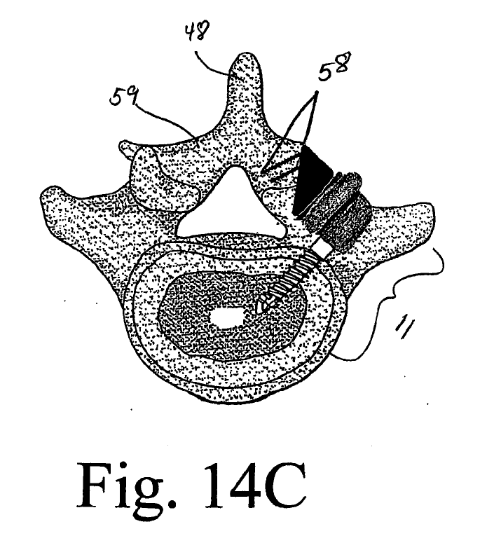

FIG. 14A demonstrates a transaxial view of the prosthetic device (11) having been passed through the target pedicle (45). Securing screws (58) have been passed through the laminar component (9) of the prosthetic device (11) to secure the trailing end of the device (2) in place.

FIG. 14B demonstrates a transaxial view with an alternative embodiment of the securing screws (58). In this instance, one or more securing screws (58) has been passed through the base of the spinous process (48) to achieve bicortical purchase of the contralateral lamina (59).

FIG. 14C demonstrates a transaxial view of a prosthetic device (11) in place with an alternative embodiment of the laminar component (9). In this embodiment, a curvy linear surface (62) is secured against the lateral aspect of the lamina (59). As in the previous embodiment, screws (58) secure the device to the lamina (59) or across the base of the spinous process (Not shown in this figure).

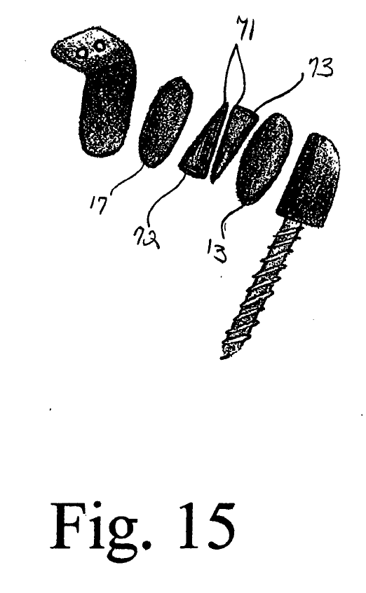

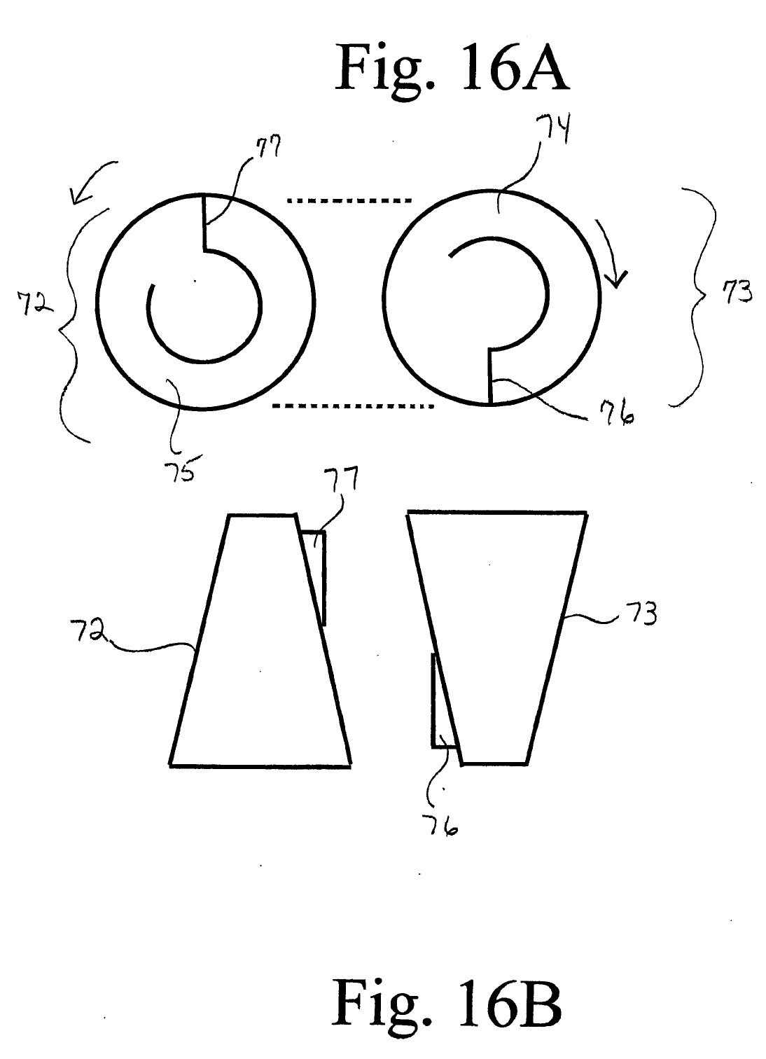

An alternative embodiment of the joint disc is also herein provided. This embodiment replaces the liquid or gel center with a mechanical joint disc (71). As seen in an exploded perspective view in FIG. 15, this is primarily composed of a medial circular component (72) and a lateral circular component (73). These are flattened and discoid in shape and are irreversibly secured to the discoid surface (13) and the discoid base (17). The medial circular component (72) and the lateral circular component (73) furthermore interface with each other. The interface between the two is composed of a spiraling ramp (see FIG. 16) on the surface of the medial circular component (72) which interfaces with a spiraling ramp (see FIG. 16) on the surface of the lateral circular component (73). These spiraling ramps are designed such that they may slide over each other as the prosthetic device (11) rotates.

In FIGS. 16A & 16B, it can be seen that each ramp (74, 75) has a termination point of maximum height (76, 77). The termination points (76, 77) are geometrically arranged such that when the motion segment is in flexion there is a maximum distance between the two points of maximum height (76, 77). Essentially this is accomplished by the fact that one ramp is spiraling “clockwise,” as seen in a disarticulated frontal perspective of the component, while the other ramp is spiraling “counter clockwise.” Furthermore, when the two circular components (72, 73) are articulated within the prosthetic device (11), the point of maximum height (76, 77) of each ramp (74, 75) is geometrically arranged so that when the motion segment is in extension, the two points of maximum height (76, 77) will interlock, preventing further extension.

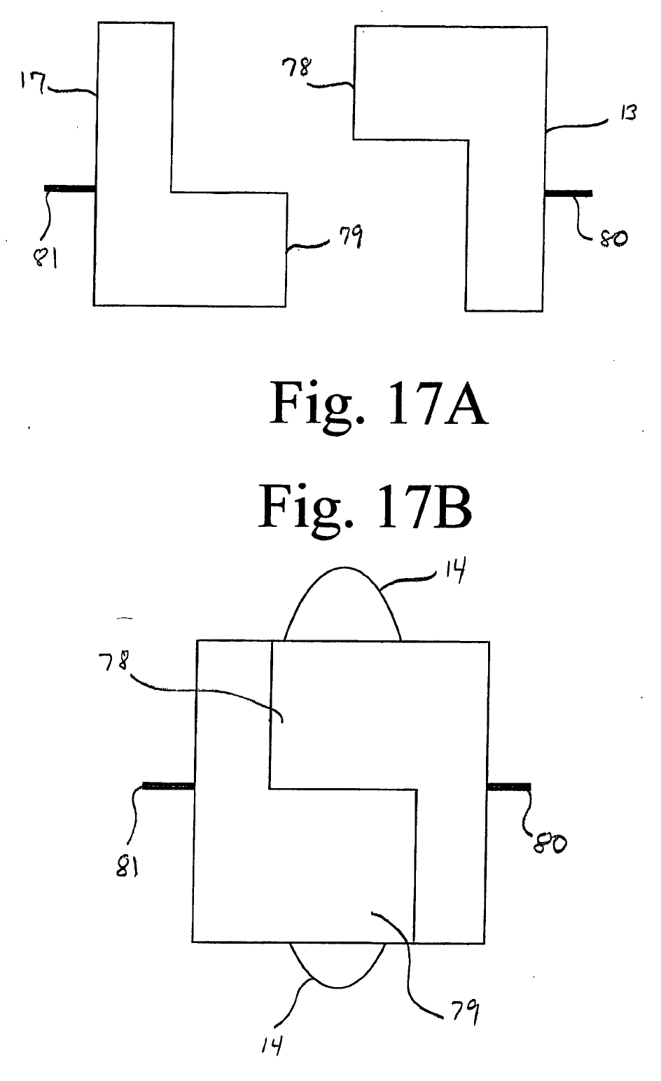

Additionally, it is to be remembered that one of the principal functions of the facet joint is to limit extension. To that end, an alternative embodiment of the discoid surface (13) and discoid base (17) is seen in FIG. 17A. In this embodiment, there is an horizontal extension (78) arising from the discoid surface (13) and a complimentary horizontal extension (79) discoid base (17). These extensions are fashioned in such a way that they will interlock if the involved motion segment attains an excessive degree of extension. A further alternative embodiment provides for a pivot or axel (80) to be positioned between the discoid surface (13) and the superior articular replacement (3) on the lateral side of the trailing end of the prosthesis (2), and a second pivot or axel (81) between the discoid base (17) and the laminar component (9) on the medial side of the trailing end (2).

While the invention has been shown and described with reference to certain preferred embodiments, it will be understood by those skilled in the arts that various changes and modifications in form and detail may be made therein without departing from the spirit and scope of the invention as defined by the appended claims.

What is claimed:

1. A system of devices and methods for use by which the natural facet joints of the spine may be replaced by a prosthetic device, the method consisting of:
identifying the location of the facet joint to be replaced using any image-guided system, including radiologic, fluoroscopic methods, laser-guided systems, computerized image-guided system, or any other image-guided system;
identifying the surface area on the skin which correlates to the subsurface structure to be replaced;
passing a needle/guide pin through a site in the skin to a point in or near the facet joint to be replaced and verifying the location of the leading end of the needle/guide pin using an image-guided system;
passing a series of dilators over the guide pin;
passing an elliptical, circular or ovoid working channel into the operative field centered over the facet joint to be replaced;
passing a burr or other cutting instrument through the working channel and removing the facet joint to be replaced;
inserting the facet joint prosthesis.

2. The facet joint prosthesis of claim 1, which is a unitized prosthesis consisting of a leading end and a trailing end.

3. The leading end of the prosthesis in claims 1 and 2 consisting of a threaded or non-threaded shaft that shall have a leading end, a shaft and a trailing end and shall be of a size, shape and pitch that can be placed within a pedicle of a vertebra of a patient.

4. The leading end of the threaded or non-threaded shaft in claim 3, which may be fashioned in such a way that it may be self-tapping, self-drilling and may be cannulated so that it can be inserted over a guide wire.

5. An alternative embodiment of the threaded or non-threaded shaft in claim 3 in which it is passed with the use of a drill and/or tap.

6. An alternative embodiment of the threaded or non-threaded shaft in claim 3 which is not cannulated and is placed into an area in the bone prepared by a trocar.

7. The trailing end of the threaded or non-threaded shaft in claim 3 which is fashioned in such as way that it can accommodate a Phillips type screwdriver, a regular screwdriver, an Allen wrench or any other similar drive mechanism.

8. The trailing end of the prosthetic device in claims 1 and 2, in which is itself composed of multiple components, namely:
an oblong or rhomboid shaped structure herein known as a superior articular replacement;
a discoid shaped area on the medial side of the trailing end of the threaded or non-threaded shaft, this discoid area serving as a base or anchor for the lateral aspect of the ballotable chamber component of the prosthesis;
a ballotable chamber composed of a layer of latex, polyester, or any other natural or synthetic material, either used or not used in related art, incasing any fluid or gas;
a discoid shaped area on the medial side of the ballotable chamber component of the prosthesis;
an area on the medial aspect of the second discoid structure to serve as a connection to the lateral aspect of the lamina, herein known as the laminar component

9. The needle/guide pin in claims 1 and 3 above, being of sufficient length, caliber, and firmness to be passed into the facet joint in accomplishing image-guided localization of the facet joints.

10. In accordance with claim 1, a series of dilators that shall be passed in a graduated order, passing the dilator with the most narrow diameter initially, and then passing successively larger diameter dilators

11. An alternative embodiment of the scheme of dilators in claim 10, by which the outermost dilator is passed initially, and successive dilators are passed through a central aperture and expanded' from in a centrifugal fashion.

12. In accordance with claim 1, a working channel, consisting of a leading end, a shaft and a trailing end, which is passed over the outside of the outermost dilator, regardless of the dilator scheme utilized.

13. The working channel in claim 12, consisting of an elliptical, ovoid or any other shape channel component of a tubular cannula that is passed into position and shall be positioned, in its functional position, with the leading end against the posterior aspect of the facet joint to be replaced and the trailing end providing the surgeon with access to the area to be operated upon.

14. In accordance with claim 1, a means of removing the target facet joint, consisting either of a cutting burr, a bone chisel of any configuration, or any other means; this may be powered by either manual, solenoid, battery, pneumatic, electric or hydraulic.

15. In accordance with claim 1, any means by which the leading end of the prosthesis may be successfully passed into the target pedicle.

16. In accordance with claim 1, a means of inserting the prosthesis consisting of passage of a wire into the pedicle after removal of the target facet joint, followed by passage of the prosthesis over the wire through a cannulated portion of the screw component of the prosthesis.

17. In accordance with claim 5, an alternative method of insertion of the prosthesis consisting of the use of a drill and tap to prepare the pedicle to receive the leading end of the threaded or non-threaded shaft.

18. A means of fastening the laminar component of the prosthesis to the lateral lamina; this means consisting of screws, pins, nails, or any other fashion by which the laminar component may be herein attached.

19. An alternative embodiment of the ballottable joint disc in claim 8, consisting of a pair of conjoined, mirror-image, disc-shaped components; each component herein having a spiraling ramp on the surface at the mutual interface.

20. The arrangement of the ramps in claim 19 is such that the ramps will interlock at their point of maximum height when the motion segment is at the physiologic limit of extension, thus functioning to limit extension.

21. A kit, comprising the methods in claim 1, consisting of a sterile transparent sheet to be laid upon the back of the patient, and a localizing needle, to be used in conjunction with the transparency in accomplishing needle localization of the facet joint with radiographic means.
